(19) United States
(12) Patent Application Publication
(10) Pub. No.: US 2016/0029931 A1
(43) Pub. Date: Feb. 4, 2016

(54) METHOD AND SYSTEM FOR PROCESSING AND ANALYZING ANALYTE SENSOR SIGNALS

(71) Applicant: Sano Intelligence, Inc., San Francisco, CA (US)

(72) Inventors: Rebeca Salas-Boni, San Francisco, CA (US); Matthew Chapman, San Francisco, CA (US); Abhijit Ghosh, San Francisco, CA (US); Ashwin Pushpala, San Francisco, CA (US)

(21) Appl. No.: 14/812,288

(22) Filed: Jul. 29, 2015

Related U.S. Application Data
(60) Provisional application No. 62/031,266, filed on Jul. 31, 2014.
(60) Provisional application No. 62/086,797, filed on Dec. 3, 2014.

(51) Int. Cl. A61B 5/1495; A61B 5/01; A61B 5/1473; A61B 5/145; A61B 5/00

(52) U.S. Cl. CPC A61B 5/1495; A61B 5/1451; A61B 5/14532; A61B 5/4812; A61B 5/4866; A61B 5/486; A61B 5/14735; A61B 5/7203; A61B 5/01; A61B 5/7275; A61B 5/7282; A61B 5/0004; A61B 2560/0223

(57) ABSTRACT

A method and system for near-real time and continuous analyte monitoring, the method including: receiving a signal stream associated with an analyte parameter of the user across a set of time points; generating a dataset indicative of values of the analyte parameter across the set of time points, upon processing of the signal stream in near-real time; performing a calibration operation on values of the analyte parameter, based upon a calibration event, thereby generating a set of calibrated values of the analyte parameter; at the computing system, identifying a set of activity events of the user, from a supplemental dataset, during a time window corresponding to the set of time points; generating an analysis indicative of an association between at least one of the set of activity events and the set of calibrated values of the analyte parameter; and rendering information derived from the analysis to the user.

### CROSS-REFERENCE TO RELATED APPLICATIONS

This application claims the benefit of U.S. Provisional Application Ser. No. 62/031,266, filed on 31 Jul. 2014, and U.S. Provisional Application Ser. No. 62/086,797, filed on 3 Dec. 2014, which are each incorporated herein in its entirety by this reference.

### TECHNICAL FIELD

This invention relates generally to the biosignal detection field, and more specifically to a new and useful method and system for processing and analyzing analyte sensor signals.

### BACKGROUND

Biomonitoring devices are commonly used, particularly by health-conscious individuals and individuals diagnosed with ailments, to monitor various biosignals. Conventional biomonitoring devices can perform the tasks of determining an analyte level in a user's body and providing information regarding the analyte level to a user; however, such biomonitoring devices typically convey information to users that is limited in usefulness, intermittent, and prompted by the command of the user. Such biomonitoring devices are also inappropriate for many applications outside of intermittent use, due to design and manufacture considerations. Additionally, current devices are configured to analyze one or a limited number of biosignal parameters (e.g., analyte parameters characterizing a physiological state of the user), due to limitations of sensors and/or processing methods used in current biomonitoring devices. Current systems and methods are further unable to detect, respond to, and correct signal artifacts in near-real time (or substantially in real time), require extensive user involvement in calibration of sensor data, and are limited in many other ways. As such, processing and analyzing such biosignal information detected using current devices and methods is inadequate in many ways.

There is thus a need in the biosignal detection field to create a new and useful method and system for processing and analyzing analyte sensor signals. This invention provides such a new and useful method and system.

### BRIEF DESCRIPTION OF THE FIGURES

FIG. 1 depicts an embodiment of a method for processing and analyzing analyte sensor signals;

FIG. 2 depicts an embodiment of a system for processing and analyzing analyte sensor signals

FIGS. 3A and 3B depict variations of processing flow in an embodiment of a method for processing and analyzing analyte sensor signals;

FIG. 4 depicts a variation of a portion of a method for processing and analyzing analyte sensor signals;

FIGS. 5A-5D depict examples of artifacts in an embodiment of a method for processing and analyzing analyte sensor signals;

FIG. 6 depicts a frequency distribution of analyte parameter values in relation to a mixture model used in an embodiment of a method for processing and analyzing analyte sensor signals; and

FIGS. 7A-7B depicts variations of steps in an embodiment of a method for processing and analyzing analyte sensor signals.

### DESCRIPTION OF THE PREFERRED EMBODIMENTS

The following description of the preferred embodiments of the invention is not intended to limit the invention to these preferred embodiments, but rather to enable any person skilled in the art to make and use this invention.

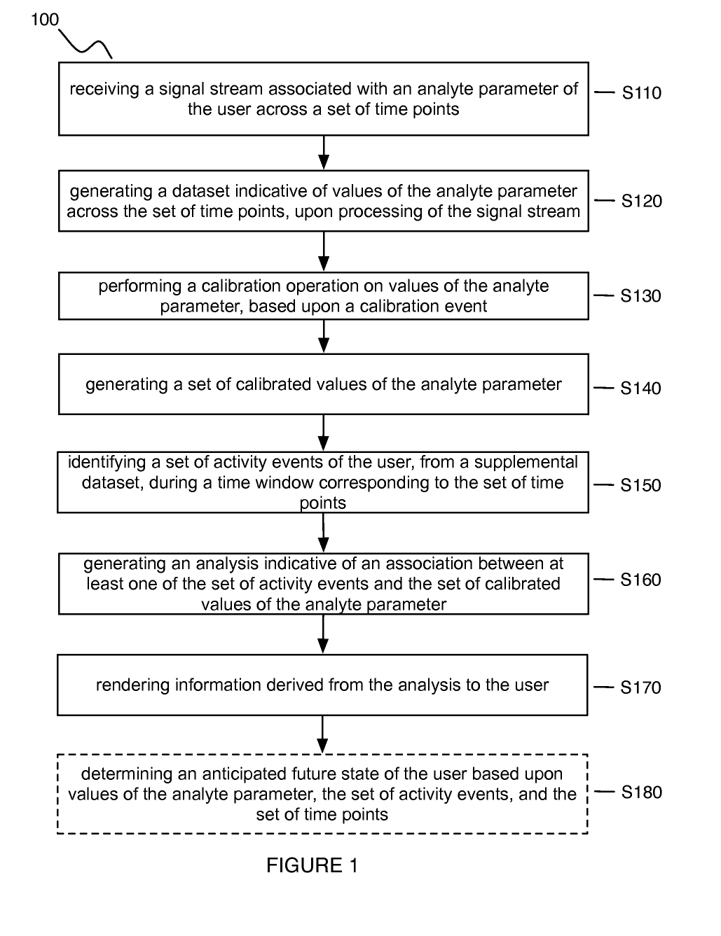

As shown in FIG. 1, an embodiment of a method 100 for processing and analyzing analyte sensor signals comprises: receiving a signal stream associated with an analyte parameter of the user across a set of time points S110; generating a dataset indicative of values of the analyte parameter across the set of time points, upon processing of the signal stream S120; performing a calibration operation on values of the analyte parameter, based upon a calibration event S130, thereby generating a set of calibrated values of the analyte parameter S140; identifying a set of activity events of the user, from a supplemental dataset, during a time window corresponding to the set of time points S150; generating an analysis indicative of an association between at least one of the set of activity events and the set of calibrated values of the analyte parameter S160; and rendering information derived from the analysis to the user S170. The method 100 can further include Block S180, which recites: determining an anticipated future state of the user based upon values of the analyte parameter, the set of activity events, and the set of time points.

The method 100 functions to facilitate monitoring of analyte sensor-derived biosignals of a user, and in preferred applications, can provide insights into a user's current and future physiological state based upon one or more detected analyte parameters. Variations of the method 100 can, however, be adapted to processing of non-analyte-derived biosignals of a user, as associated with any other suitable signal stream. In some embodiments, the method 100 can further assist the user in modifying his or her behavior to adjust his/her physiological state in a desired manner, thereby improving the user's health. As such, the user can be provided with insights into the effect(s) of his or her behavior on his/her physiological state, as indicated in measured analyte parameters, in order to affect a behavior change in the user. Variations of the method 100 can further process received signals in order to determine an anticipated future state of the individual, and provide notifications configured to prevent a transition into an adverse physiological state, to promote a transition into a positive physiological state, or to maintain a desired current state.

The method 100 is preferably implemented at least in part with an analyte sensor configured to access and measure one or more analyte parameters from a body fluid of the user, and in some embodiments, can implement an embodiment of the sensor described in U.S. application Ser. No. 14/211,376, entitled “On-Body Microsensor for Biomonitoring” and filed on 14 Mar. 2014, which is herein incorporated in its entirety by this reference. As such, the analyte sensor can be a continuous analyte monitor configured to be worn by a user for an extended period of time (e.g., one week, weeks, one month, etc.).

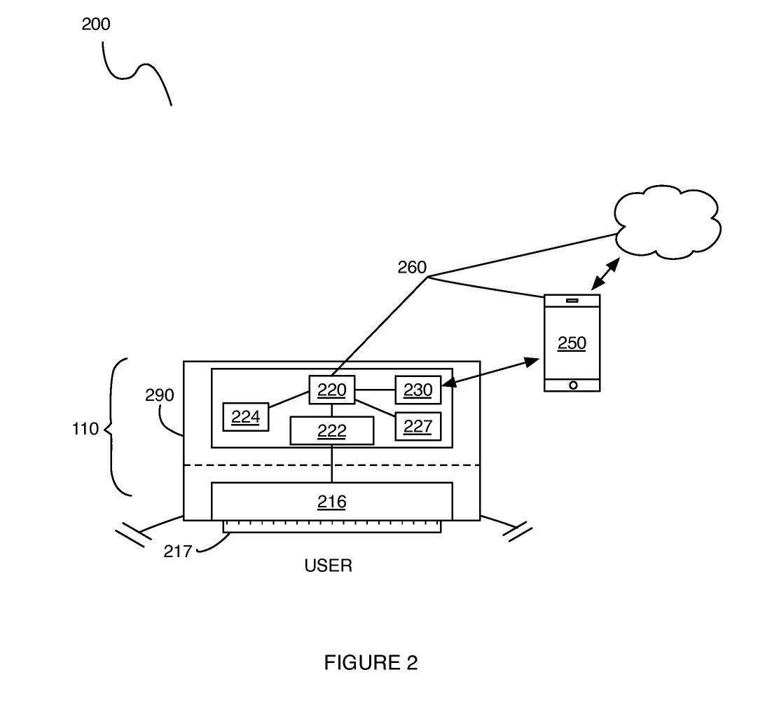

Additionally or alternatively, at least a portion of the method 100 can be implemented using an embodiment, variation, or example of the system described in U.S. application Ser. No. 14/657,973, entitled “System for Monitoring Body Chemistry” and filed on 13 Mar. 2015, which is herein incorporated in its entirety by this reference. As such, in one embodiment (as shown in FIG. 2), a system 200 including an analyte sensor 210 and a computing system 260 implementing at least a portion of the method 100 can include: a housing 290 that supports a microsensor 216 including an array of filaments 217, and an electronics subsystem 220 in communication with the microsensor 216; and a processing subsystem 260 configured to perform at least a portion of the method 100. In more detail, the housing 290, microsensor 216, and the electronics subsystem 220 can be configured as an analyte sensor 210 configured to sense analyte levels in a user's body, wherein the electronics subsystem includes a signal conditioning module 222, a power management module 224, a storage module 227, and a transmitting unit 230 in communication with the computing system 260 and/or an electronic device (e.g., mobile computing device 250) associated with the user.

However, variations of the method 100 can additionally or alternatively be implemented, at least in part, using any other suitable sensor system configured to detect biosignals of the user. Furthermore, the method 100 can be implemented, at least in part, using an application (e.g., mobile device application, web application, etc.) executing at an electronic device (e.g., personal computer, mobile device, wearable computing device, etc.) of the user, such that the user can be provided with insights related to analyses produced using the method 100.

### 1. Method

Block S110 recites: receiving a signal stream associated with an analyte parameter of the user across a set of time points, which functions to enable detection of analyte-derived signals detected in body fluid of a user. Block S110 further functions to enable determination of values of one or more analyte parameters characterizing a physiological state of the user over time, in order to generate biometric data from which insights can be derived in subsequent blocks of the method 100. Block S110 is preferably implemented by way of an analyte sensor configured to interface with body fluid of the user during operation; however, Block S110 can alternatively be implemented using any other suitable biometric signal configured to detect and/or transmit signal streams associated with one or more physiological processes of the user. In some embodiments, Block S110 can be implemented at least in part with an embodiment, variation, or example of the microsensor described in U.S. application Ser. No. 14/211,376, entitled “On-Body Microsensor for Biomonitoring” and/or the system described in U.S. application Ser. No. 14/657,973, entitled “System for Monitoring Body Chemistry”, which is configured to interface with interstitial fluid of the user; however, the analyte sensor of Block S110 can additionally or alternatively be configured to access any other suitable body fluid of the user.

As such in some embodiments, the analyte sensor implemented in Block Silo can comprise an electrode module comprising one or more working electrodes, one or more counter electrodes, and one or more reference electrodes configured to facilitate analyte sensing from a body fluid of the user. However, Block S110 can additionally or alternatively be implemented, at least in part, using any appropriate analyte parameter sensor, and/or the electrode module can include any one or more of an offset, unbalanced, mutually biased, and mutually unbiased electrode. Block S110 can include sensing multiple analyte parameters (e.g., parameters of body glucose, potassium, sodium, calcium, carbon dioxide, nitrogen, iodide, iodine, etc.); however, Block S110 can alternatively include sensing a parameter for a single analyte of interest.

Preferably, the set of time points associated with the signal stream received in Block S110 includes uniformly spaced time intervals (i.e., with uniform frequency), such that values of the analyte parameter determined from the signal stream can be associated with regularly spaced time points (e.g., every minute, every day, every week); however, the set of time points can additionally or alternatively include time points relevant to a user based upon user activity (e.g., meal times, exercise periods, rest periods, medication times, etc.), based on user prompting (e.g., the user can indicate when he or she would prefer analyte parameter measurement), based upon prompting by any entity (e.g., a caretaker of the user), and/or based upon any other suitable factor. As such, the set of time points can be irregularly and/or randomly spaced, with sparsity of data points bound by theoretical limits (e.g., as determined using convex optimization, etc.).

In some variations, generation/reception of the signal stream at the analyte sensor, for measurement of values of analyte parameter(s), can additionally be triggered (e.g., based upon automatic detection of an event of the user). For instance, signal reception can be automatically triggered upon detection that the user has begun a period of exercise, by way of a motion sensor (e.g., accelerometer, gyroscope) that detects motions of the user. In another example, generation/reception of the signal stream for measurement of values of analyte parameter(s) can be automatically triggered upon detection that the user has entered a dining facility, by way of a location sensor (e.g., global positioning device, triangulation system, etc.) that identifies locations of the user. In another example, generation/reception of the signal stream for measurement of values of analyte parameter(s) can be automatically triggered upon detection that the user has had a “significant” measurement of biometric parameter (e.g., analyte parameter, heart rate, body temperature, respiration parameter, etc.) as measured by a supplementary sensing system (e.g., of another device, of the same device, by way of an API, etc.), wherein variations of supplemental datasets supporting the method 100 are described in further detail below. In another example, social context information from a user (e.g., as associated with electronic social network check-ins, status updates, tagging events that associate the user with another entity, place, or activity, etc.) can automatically trigger signal acquisition/provision. In another example, entering proximity to a device (e.g., a mobile phone, a home appliance) or signal (e.g., active signal, passive signal, visual pattern signal, electrical signal, sonic signal, etc.) can automatically trigger measurement or detection of an analyte parameter.

However, signal stream generation and reception, for determination of values of the analyte parameter(s) in subsequent blocks of the method 100 can additionally or alternatively include manual triggering or no triggering in association with one or more time points of the set of time points. Additionally, the set of time points may be set automatically, or may be adjusted to include or omit time points as prompted by a user.

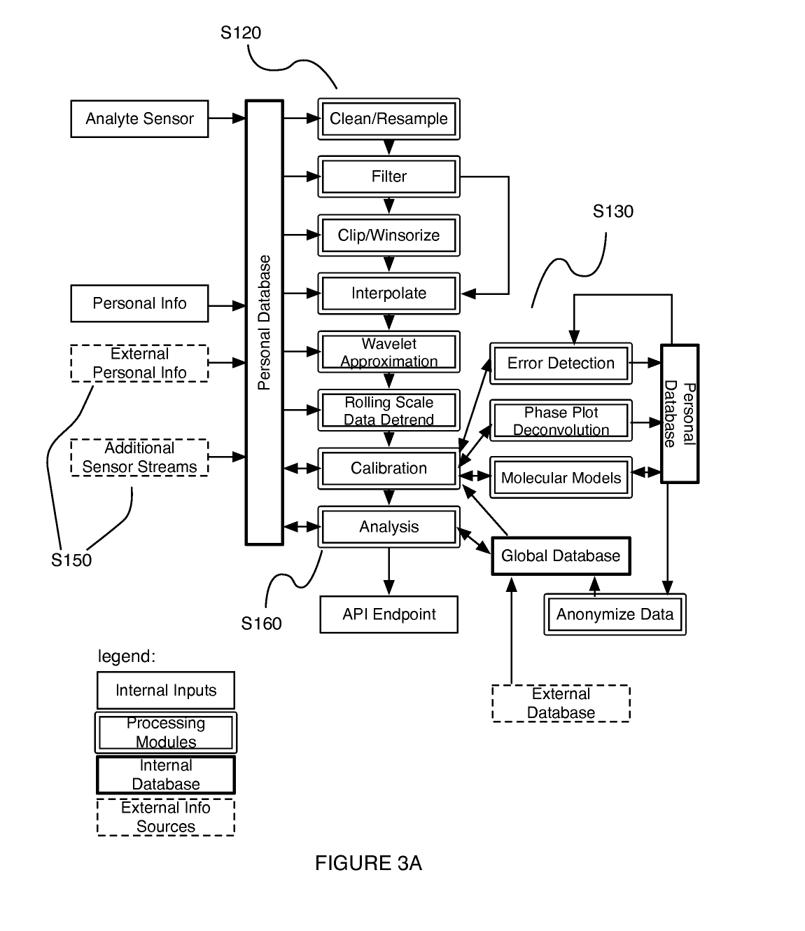

As shown in FIG. 3A, the signal stream from the analyte sensor can be transmitted to an/or stored at a personal database, wherein the personal database in an internally maintained repository for all personal information and sensor data associated with a particular user. Data within the repository can then be transmitted to other modules of a system associated with the method 100, for subsequent processing, as described in more detail below.

### 1.1 Method—Signal Processing

Block S120 recites: generating a dataset indicative of values of the analyte parameter across the set of time points, upon processing of the signal stream, which functions to process data derived from the signal stream of Block S110 according to at least one data conditioning algorithm. Block S120 is preferably implemented at a computing system in communication with the analyte sensor of Block S110, wherein the computing system can be implemented in one or more of: a remote server, a personal computer, a cloud-based computing system, a computing module of a mobile electronic device (e.g., mobile communication device, wearable computing device, etc.), and any other suitable computing system. Furthermore, in relation to Block S110, the computing system can host or otherwise be associated with the personal database/repository configured for reception, storage, and transmission of data associated with a user. Preferably, Step S120 includes transforming an electrical signal (e.g., voltage, current impedance, or capacitance) produced by the analyte sensor, in response to a sensed analyte, into a derivative parameter (e.g., a level of a parameter in body fluid of the user) in a streaming manner, through variations of the data processing pipeline described below. The output(s) of Block S120 is/are preferably a dataset characterizing one or more values associated with the analyte parameter(s) over the set of time points, and in some variations can include concentrations of one or more analytes characterized over time; however, the output of Block S120 can alternatively be any other suitable output. Preferably, Block S120 transforms measurements detected at the analyte sensor of Block S110 into a conditioned dataset that can be used to determine parameter values as in Block S140 in a robust manner.

As described in more detail below, signal processing in Block S120 preferably accounts for at least the following: physiological limitations (e.g., what analyte parameter values and changes in analyte parameter values are possible in the body), population distribution factors (e.g., effects of age, gender, and other demographic information on analyte parameter values), analyte sensor equilibration (i.e., sensor stabilization upon coupling of the sensor to the user), signal degradation (e.g., in response to sensor signal changes over time of use by a user), diurnal variation in analyte levels, signal latency (e.g., in relation to differences between analyte detection from blood vs. from interstitial fluid), and other factors.

In some variations, as shown in FIGS. 3A and 3B, Block S120 can include preprocessing the signal stream of Block S110, which functions to ensure signal stream quality prior to further processing in Block S120. In some variations preprocessing the signal stream can include performing a screening step to ensure that there are no missing data points, that the sensor readings have continuous time stamps (i.e., there is no lapse in the signal stream), that the time stamps are accurate, and that there are no error notifications (e.g., error codes) associated with configured error detection settings of the analyte sensor. Preprocessing in Block S120 can, however, include any other suitable preprocessing step.

In relation to processing of the signal stream (e.g., after performance of a preprocessing operation), Block S120 can include any one or more of: denoising, filtering, smoothing, clipping/winsorizing, transformation of discrete points to a continuous function, deconvolving, standardizing, detrending, resampling, and performing any other suitable data conditioning process upon raw biosignal data received in Block S110. For instance, some variations of Block S120 can additionally include performing a prediction operation, performing a windowing operation, and/or performing a signal cleaning operation. In more detail, predicting can include performing a filtering operation on biosignal data up to a time (t), and applying the operation to additional data points up to time (tₙ) in the future. In more detail, windowing can include delay-free windowing methods (approximated on long time intervals spanning signal detection), and in specific examples, can include any one or more of moving windows, exponentially weighted moving windows (EWMW), moving Gaussian methods, Parzen methods, and any other suitable window filtering method. In more detail, signal cleaning can include removal of spurious signals (e.g., as defined by physiological and/or physical limits) by way of a comb filter derived from physical properties of analyte parameter dynamics (e.g., glucose dynamics) and/or any other suitable algorithm, variations of which are described in Section 1.1.1 below.

In variations, filtering can include signal processing up to a time (t), which can be performed in real-time, while smoothing can include signal processing over past, present, and/or future data (e.g., over an entire biosignal dataset), which can be implemented using delayed signal processing and/or offline signal processing. In specific examples, Kalman filter and smoothing implementations of state space models of system zero-, first-, and second-order dynamics can be implemented, including any one or more of: nonlinear Kalman filter methods (e.g., extended Kalman filter, unscented Kalman filter); bootstrap fillers, particle filters (e.g., expectation maximation methods, Markov Chain Monte Carlo methods, metropolis-Hasting methods, simulated Annealing methods, nonlinear multilayer perceptron, artificial neural networks, nonlinear machine learning modules, etc.) that rely upon Bayesian Networks (e.g., a variational Bayes approximation); peak finding methods (e.g., slope-based, wavelet-based, etc.) to locate maxima and minima, to facilitate time-delay estimation (e.g., from diffusion limited blood analyte to interstitial analyte levels), to facilitate data correction (e.g., using deconvolution, using state-space approaches, etc.); and any other suitable method. In addition to or alternative to state space models, any one or more of: Continuous Random Field approaches, Perturbation approaches, Gauge Variance approaches, field approaches, Erdogic approaches, thermodynamic approaches, complexity approaches, and any other suitable approach translated form Lagrangian, Hamiltonian, Quantum, Statistical Mechanics, and other physical theories (e.g., Brownian, Wiener Processes, Levy Flight, Complex Adaptive Systems, etc.) can be implemented in signal processing.

In more detail associated with one variation of filtering, as shown in the flowcharts of FIGS. 3A and 3B, short windows of time associated with the signal stream can be resampled in Block S120, and one or more of a modified Markov Chain Monte Carlo Particle Filter, a Laplace approximation, and a variational Bayes Approximation can be applied to compute a probability for various positions and trajectories of the analyte sensor output at each point of the sampled window. This process functions to remove outliers within the data and to smooth the data to facilitate implementation of subsequent processing stages (e.g., stages of interpolation, etc.). In relation for computed positions/trajectories at this stage, personal data (e.g., historical data, health information, contextual data, other forms of data described in more detail below, etc.) from the user, as determined from data within the personal database/repository described above, can be used to place limits on rates of change in the analyte sensor output based upon a feasibility assessment.

In more detail associated with one variation of clipping/winsorizing, as shown in the flowchart of FIG. 3A, Block S120 can include performing one or more statistical transformations that place limiting thresholds on analyte sensor outputs values (i.e., extreme values), thereby clipping aberrant sensor responses due to moisture (e.g., due to sweat, due to exercise, etc.), mechanical motion (e.g., of the analyte sensor relative to the body of the user), and any other suitable transient noise source. To preserve data continuity, a faded clipping operation implementing one or more of a Huber filter, an M estimator, and any other suitable statistical estimator can be used instead of a process that performs a hard cut off. Threshold limits used in a clipping/winsorizing operation can be based upon one or more of: expected analyte sensor output ranges and distributions of output values and information from the personal database of the user (e.g., historical information, health information, risk factors, etc.).

In more detail associated with one variation of transformation of discrete data points into a continuous function, as shown in the flowchart of FIG. 3A, Block S120 can include performing an interpolation operation including one or more of: a spline (e.g., a b-spline interpolation), a Bessel function interpolation, a path integral operation (e.g., a Feynman-Kac path integral operation), and any other suitable interpolation operation. The interpolation operation can thus support a wavelet approximation operation, which requires a continuous function, as described in further detail below. Limits similar to those used in the filtering operation described above (e.g., the Bootstrap filtering operation) can be used to restrict the resultant interpolation/path integral to ensure accuracy of the operation in relation to realistic physiological responses. For instance, the resultant interpolation model can be bound by physiologically relevant blood glucose concentrations and/or other biophysical parameters that are feasible for a given window of time.

As indicated above and shown in the flowchart of FIG. 3A, Block S120 can include conditioning raw biosignal data based upon a wavelet approximation algorithm, which functions to analyze rapidly changing transient signals (e.g., in real time, in near-real time), such as those detected at the analyte sensor, in terms of a set of wavelet basis functions that can be scaled and/or translated, while still retaining localization in the time and the frequency domain. As such, the wavelet approximation algorithm functions to provide further smoothing and detrending of data from the analyte sensor without distorting the underlying signal or introducing a time lag component. In variations, the wavelet basis functions can include any one or more of: Daubechies wavelets, Dual-Tree Complex wavelets, Haar wavelets, undecimated wavelets, Newland wavelets, and any other suitable wavelet basis function(s). Furthermore, in variations, the wavelet analysis can implement a continuous wavelet transform that can construct a time-frequency representation of a signal. In one application of Block S120, generating the biosignal dataset can implement a continuous wavelet multi-resolution analysis that isolates, filters and/or processes raw biosignal data to produce a conditioned dataset with an appropriate time scale. However, any other suitable conditioning process, such as a fast Fourier transform (FFT) process, can additionally or alternatively be used to process raw biosignal data in Block S120.

Post-implementation of the wavelet approximation algorithm, Block S120 can include performance of a rolling scale detrending operation, as shown in the flowchart of FIG. 3A. The rolling scale detrending operation can remove sensor drift from data derived from the signal stream by observing the mean and standard deviation of rolling windows of sensor data of different time scales. By using discrete time intervals, the data associated with each time interval is locally rescaled in a manner that reduces sensitivity of the processing methods in Block S120 to erroneous peaks. The rescaling process can be applied over physiologically relevant time scales, which can prevent erroneous peaks from affecting the scale of peaks through the entire dataset associated with the signal stream. In particular, one application of the rolling scale detrending operation can be used to retrieve relevant analyte-associated (e.g., glucose-associated) signals from the signal stream, while rejecting fluctuations due to noise or sensor drift with a high level of discrimination. However, any other suitable detrending operation can be performed in Block S120, some additional variations of which are described in Section 1.1.1 below.

In supporting removal of noise during signal processing, Block S120 can also implement signals of a blank electrode, associated with the analyte sensor of Block S110, to facilitate detrending and/or noise reduction or elimination from raw biosignal data of the sensing electrode module. In variations, the blank electrode can be included on-chip with the analyte sensor of Block S110, or can alternatively be associated with the analyte sensor in any other suitable manner. For instance, a batch of analyte sensors can have an associated blank electrode that can be used to provide a noise signal indicative of measurement noise for the batch of analyte sensors. In another example, a blank electrode can be paired with each analyte sensor (e.g., in embodiments wherein a set of analyte sensors coupled to a user is configured to detect a set of different analyte types) The blank electrode is preferably identical to a working electrode of the sensing electrode module in morphology, but does not include molecules or particles (e.g., complementary enzymes) that facilitate sensing of the analyte parameter(s). However, the blank electrode can alternatively be any other suitable electrode that serves as a control. As such, in variations of Block S120, an artifact signal (i.e., a signal that represents signal noise and/or drift) can be used to eliminate signal artifacts (e.g., noise, drift), from raw biosignal data during conditioning to produce the biosignal dataset.

Block S120 can additionally or alternatively include any other suitable signal-processing step that facilitates generation of the biosignal dataset. In some variations, Block S120 can implement probabilistic models (e.g., Multi-Arm Bandit approaches) to facilitate determination of a value of a sensor signal among a discrete set of possible values, and/or methods for signal source separation (e.g., independent component analysis). Additionally or alternatively, Block S120 can include implementing a machine-learning algorithm or other algorithm configured to facilitate automatic identification of features (e.g., characteristic physiological responses, outliers) within raw or processed biosignal data. The machine learning algorithm(s) can include any suitable class of machine learning algorithm (e.g., unsupervised learning, semi-supervised learning, transduction, reinforcement learning, etc.) and can additionally or alternatively involve implementation of a data-mining algorithm. Any models produced using a machine learning algorithm can further be trained with data from the user and/or data from any other user(s) to increase the robustness of analyses derived from outputs of Block S120.

### 1.1.1 Signal Processing—Artifact Detection

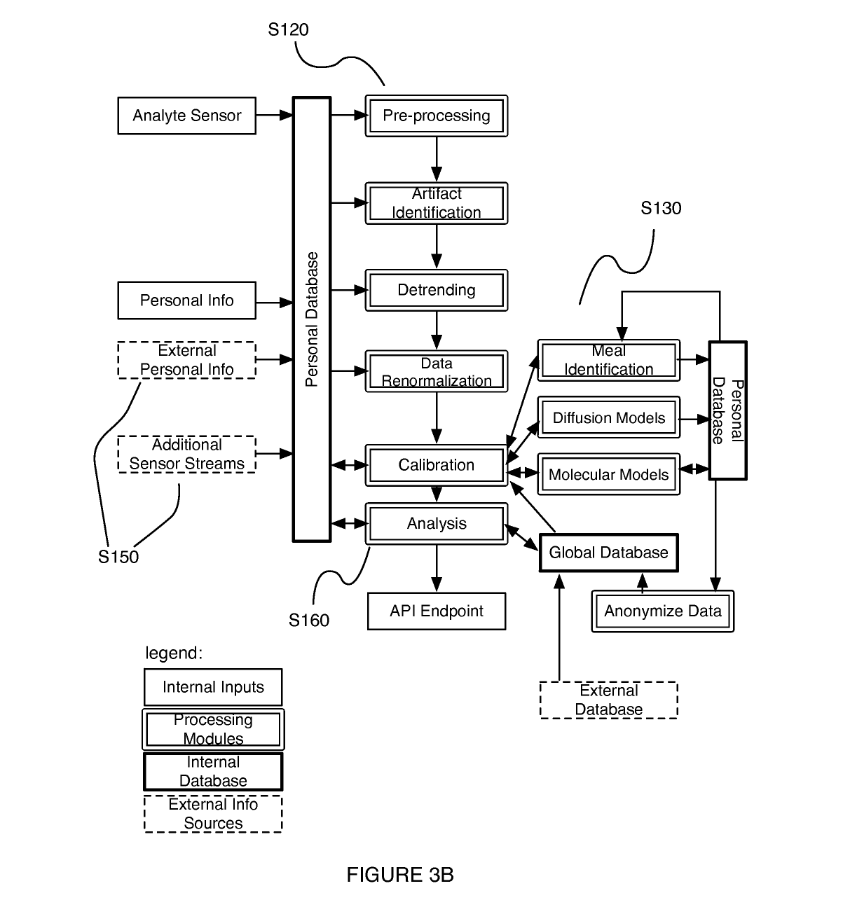

In relation to signal processing steps described above, Block S120 can include blocks configured for detection of an artifact (e.g., spurious noise, peak, etc.) and correction of the artifact in near-real time to produce a dataset indicative of values of the analyte parameter over time. As shown in FIG. 4, one variation of artifact processing can include: detecting an artifact within the signal stream in near-real time S121; performing a characterization of the artifact as one of a short duration artifact and a long duration artifact S122; and performing a correction operation on the artifact according to the characterization in near-real time S123.

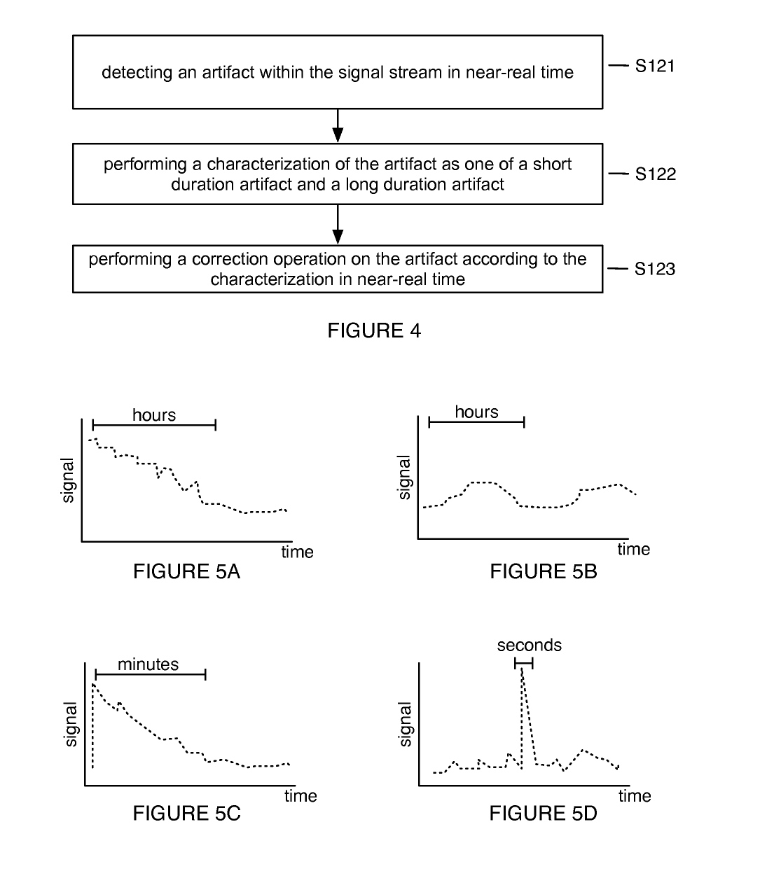

In relation to Block S121, detecting an artifact within the signal stream in near-real time can comprise detecting artifacts associated with one or more of: noise, sensor equilibration, sensor drift, environmental effects (e.g., moisture, physical disturbance of the analyte sensor, temperature, etc.), physiological cycles, spurious values, peaks, and any other suitable type of signal artifact. In relation to sensor equilibration and sensor drift, a signal artifact can be associated with a time scale of hours (e.g., 2-5 hours) over which the signal stream reaches an equilibrium state, as shown in FIGS. 5A and 5B. In relation to environmental effects, such as physical disturbance of the analyte sensor (e.g., the analyte sensor experiences an impact), a signal artifact can be associated with a time scale of minutes (e.g., 5-30 minutes) over which the signal stream recovers, as shown in FIG. 5C. In relation to spurious peaks, a signal artifact can be associated with a time scale of seconds, as shown in FIG. 5D. However, any other suitable artifact detected in Block 5221 can be associated with any other suitable time scale.

In one variation of Block S121, artifact detection can be performed on the signal stream upon filtering the signal stream with a median filter and an infinite impulse response (IIR) filter, wherein the IIR filter is characterized by the following expression: yᵢ=yᵢ₋₁+a(xᵢ−yᵢ₋₁), where a is selected to provide a lowpass filter for the desired frequency of the IIR filter. However, the parameter a can be selected to provide any other suitable filter for any other desired frequency. A rolling mean determined from a large window of data is then subtracted from the signal stream, in order to mitigate sensor drift effects. Peak detection is then performed by computation of successive differences in the filtered signal, and comparison of the difference between an incoming sample of the signal stream and a previous sample of the signal stream to an online adaptive threshold. This peak detection operation is preferably performed in near-real time as the signal stream is received, such that peaks are detected and corrected in near-real time; however, peak detection in Block S121 can additionally or alternatively be performed according to any other suitable algorithm, and/or in non-real time (e.g., in post-processing of a signal).

In Block S122, performing a characterization of the artifact as one of a short duration artifact and a long duration artifact functions to facilitate identification of an appropriate artifact correction algorithm that can be used to correct the artifacts/peaks detected in near-real time. In Block S121, the short duration artifact can be associated with one or more of: a physical disturbance of the analyte sensor (e.g., the analyte sensor experiences an impact), an environmental effect, spurious noise, and any other suitable cause of a short duration artifact. The long duration artifact can be associated with one or more of: sensor equilibration (e.g., upon application of the analyte sensor to the user), sensor drift, effects of environmental temperature, effects of environmental moisture, and any other suitable cause of a long duration artifact.

In Block S122, the characterization can be performed according to a time duration-based characterization, wherein artifacts having an associated time scale above a certain time threshold are characterized as a long duration artifact, and artifacts having an associated time scale below a certain time threshold are characterized as a short duration artifact. The characterization can additionally or alternatively be performed according to a signal signature-based characterization, wherein artifacts having a first type of signature profile (e.g., a profile associated with a first parametric function) are characterized as a short duration artifact, and artifacts having a second type of signature profile (e.g., a profile associated with a second parametric function) are characterized as a long duration artifact. In a specific example, a short duration artifact can be characterized as an artifact having a time scale of seconds or minutes, with an impulse-type signature, as shown in FIG. 5D, while a long duration artifact can be characterized as an artifact having a time scale of hours, with a constantly decreasing signature, or a slowly increasing and/or decreasing signature, as shown in FIGS. 5A and 5B. Artifact characterization can, however, be performed in Block S122 in any other suitable manner.

In Block S123, performing a correction operation on the artifact according to the characterization in near-real time functions to rapidly mitigate effects of artifacts in the signal stream, in producing a dataset indicative of values of the analyte parameter (e.g., glucose parameter) across a relevant time window, in near-real time. Block S123 preferably involves performing a first correction operation on a detected and characterized short duration artifact, and a second correction operation on a detected and characterized long duration artifact; however, Block S123 can additionally or alternatively involve any other suitable correction operation upon any other type of artifact detected and characterized according to Blocks S121 and S122.

In one variation, correction of a short duration artifact in Block S123 can include using a forecast from previous portions of the signal stream (e.g., in an orthogonal signal correction algorithm), and correction of a long duration artifact can include performing a parametric fit to a set of samples following the detected artifact, with subtraction of the parametric fit from the raw signal stream. However, the correction operation(s) used in Block S123 can additionally or alternatively comprise any other suitable correction operation(s).

Artifact processing and correction can, however, be performed in any other suitable manner, in order to account for signal artifacts in the signal stream in near-real time, real time, or any other suitable time (e.g., during signal post-processing). Furthermore, artifact correction can involve any shifting and/or scaling of data within the signal stream, in order to output the dataset indicative of values of the analyte parameter (e.g., glucose parameter) across a relevant time window, with appropriate magnitude and phase.

### 1.2 Method—Calibration

Block S130 recites: performing a calibration operation on values of the analyte parameter, based upon a calibration event S130, which functions to perform a multiscale calibration operation that normalizes analyte parameters determined from the signal stream in a manner that accounts for cyclical and other regular/non-regular variations in the analyte parameter (e.g., blood glucose parameter) of the user(s) associated with the method 100. For instance, cyclical variations can be associated with one or more of: circadian rhythms, hormonal cycles, human behavior, social schedules of the user(s), and any other suitable pattern at different time scales (e.g., hours, days, months, years, etc.). Block S130 can use patterns of human behavior and physiological response to derive likely analyte parameter value ranges for calibration, as described in more detail below.

In Block S130 the calibration operation can comprise application of a mathematical model to processed signal stream data to determine values of the analyte parameter. In a specific example, the analyte parameter can be a blood glucose concentration; however, the analyte parameter can additionally or alternatively comprise any other suitable parameter associated with an amount of analyte detected within body fluid of the user. In relation to calibration of a glucose sensor, the calibration operation can implement a model that describes the dynamics and responses of blood glucose concentrations in individuals based upon factors including one or more of: time of day, location of the user, consumption behavior (e.g., food consumption, drink consumption, substance consumption, etc.) of the user, activity (e.g., exercise) behavior of the user, blood test results of the user, demographics (e.g., weight demographics, age demographics, ethnicity demographics, nationality demographics, gender demographics, sexual identity demographics, etc.) of the user, clinical diagnoses (e.g., in relation to diabetes, in relation to cardiovascular disease, in relation to pregnancy, etc.) of the user 3^{rd} party sensor-derived factors, and any other suitable factor.

In Block S130, the calibration event is preferably unassociated with a blood-sampled measurement of the user. As such, the calibration is preferably performed in Block S130 without need for the user to provide a blood sample (e.g., by way of a finger stick test, by way of lancing, by undergoing a blood draw, etc.). However, alternative variations of the calibration event can additionally or alternatively comprise a blood-based measurement. In Block S130, the calibration state of the user is further preferably a state in which an analyte level of the user is substantially constant; however, the calibration state can alternatively be any other suitable state that can be used to provide baseline data. The calibration state of the user can be identified based upon a time point of the set of time points over which determination of the analyte parameter(s) occurs in Blocks S110 and S120, or can additionally or alternatively be identified according to a calibration event of the user. The baseline data of the calibration state can be determined automatically (e.g., at a time point of the set of time points, based upon automatic detection of an event of the user), or can additionally or alternatively be determined manually, as prompted by the user or any other suitable entity. In one variation, the calibration state of the user can correspond to a fasted baseline of the user, wherein the user has not consumed anything for a specified period of time (e.g., greater than 4 hours). In a specific example, the fasted baseline can be a glucose level measured from the user at a time point corresponding to an exit from a period of sleep (or any other suitable time point in relation to sleep), wherein a human user's glucose level is typically consistent day-to-day (e.g., between 70 and 90 mg/dL, approximately 80-85 mg/dL). In the specific example, exit from a period of sleep can be automatically identified (e.g., based upon motion of the user), or manually identified (e.g., upon indication by the user or another entity that the user has exited a period of sleep).

In another variation, the calibration state can correspond to a period after consumption of a consumable by the user, wherein the consumable produces a predicable physiological response in the user. In more detail associated with a specific example, the calibration state can be associated with a meal time, wherein predicted diets (e.g., predicted from historical user behavior) or known diets (e.g., determined from user inputs, determined from additional sensor streams, determined from user status updates, etc.) can be used to calibrate analyte responses based upon age, weight, location, and any other suitable state of the user that can provide features that encode changes in the morphology of the signal stream. These features can then be used to refine the sensor calibration, as shown in FIGS. 3A and 3B, in order to provide more accurate values of the analyte parameter(s) of interest. In this variation, the calibration state can automatically be identified (e.g., based upon detection through a GPS module that the user has entered a dining facility providing the consumable, based on a status update in an electronic social network, etc.) or can manually be identified upon prompting by the user when he or she consumes the consumable. For instance, the user can be prompted (e.g., using a native application executing on a mobile computing device of the user) to ingest a known substance at a specified time of day to calibrate the sensor, based upon the predicted effect of ingestion of the substance. In other variations, any other suitable event of the user (e.g., exercise event, consumption event, mental state event, etc.) can be used to identify the calibration state of the user.

Block S130 can additionally or alternatively include identifying the calibration state based upon historical biosignal data of the user. In one such variation, Block S130 can include locating a minimum (e.g., a minimum analyte level) of historical raw or processed biosignal data, wherein the minimum is indicative of a calibration state of the user. In other variations, any other peak or plateau of historical raw or processed biosignal data can be indicative of a calibration state of the user. In another example, wherein the calibration state corresponds to a substantially constant analyte level, a change (e.g., increase, decrease) or response in the analyte level surrounding a potential calibration point can be used to disregard the potential calibration point as indicative of a calibration state. In variations of this example, a minimum rate of change in an analyte parameter prior to a calibration point can be specified as a requirement for identification of the calibration point as corresponding to a calibration state. However, the calibration state can alternatively be determined in any other suitable manner. Furthermore, Block S130 can include identification of multiple calibration states for a user.

The calibration operation implemented in Block S130 can be adapted for each user associated with the method 100, based upon information from one or more of: the personal database, as shown in FIGS. 3A and 3B, a global database, error detection operations, deconvolution operations (e.g., a phase plot deconvolution), molecular models, diffusion models, and any other suitable operation or factor, as described in more detail below. Furthermore, the calibration operation can be iteratively refined using contextual information, historical data, secondary analyses, and/or any information determined from a supplemental dataset, as described in Block S150, to produce the calibrated values of Block S140.

As shown in the flowchart of FIGS. 3A and 3B, the global database implemented in association with Block S130 can be used to store and transmit anonymized (i.e., stripped of personally identifying information) data (e.g., statistics) from a population of users associated with the method 100, as aggregated from the personal databases of the population of users and/or 3^{rd} party databases (e.g., by way of application programming interfaces). As such, data from the population of users in the global database can be used to generate population-wide conclusions for different subsets of users associated with one or more of: different demographics (e.g., genders, ages, marital statuses, ethnicities, nationalities, socioeconomic statuses, sexual orientations, etc.), different health conditions (e.g., health and disease states), different living situations (e.g., living alone, living with pets, living with a significant other, living with children, etc.), different dietary habits (e.g., omnivorous, vegetarian, vegan, sugar consumption, acid consumption, etc.), different behavioral tendencies (e.g., levels of physical activity, drug use, alcohol use, etc.), different levels of mobility (e.g., related to distance traveled within a given time period), and any other suitable trait that has an effect on analyte levels. Data from the population of users can additionally or alternatively be used to identify correlations with contextual information (e.g., contextual information derived from the supplemental dataset of Block S150 described below) and/or to refine physiological models. The global database can, however, be used in any other suitable manner in relation to Block S130 and/or other blocks of the method 100.

As shown in the flowchart of FIG. 3A, error detection operations implemented in association with Block S130 can implement error notifications (e.g., error codes) associated with configured error detection settings of the analyte sensor and supporting electrode data streams other than those from a primary sensor to identify aberrant data points within the signal stream. Aberrant data points can be withheld prior to iterative refinement of the calibration, or corrected in any other suitable manner.

As shown in the flowchart of FIG. 3A, deconvolution operations implemented in association with Block S130 can include a phase plot deconvolution operation that enables the user's physiological responses and the performance of the analyte sensor to be deconvoluted. In particular, the dynamics of the data derived from the analyte sensor can provide information regarding 1) the user's unique physiological response (e.g., in relation to a diabetic vs. non-diabetic state, in relation to any other suitable physiological state) and 2) the performance of the analyte sensor independent of calibration. The deconvolution operation can implement a stability analysis to determine exponents (e.g., Hurst exponent, Lyapunov exponent, etc.) associated with a mixed parameter space, which can facilitate determination of the calibration operation used for the user's signal stream, as well as mapping of non-linearities in the response of the analyte sensor. Additionally or alternatively, analysis of dynamics of the data in phase space can be used to categorize peaks and/or signal trajectories as legitimate responses or noise. As shown in the flowchart of FIG. 3A, the deconvolution operation can be used to iteratively refine the calibration operation used in Block S130, and outputs of the deconvolution operation can be stored within the personal database of the user.

As shown in the flowchart of FIGS. 3A and 3B, molecular/physiological models implemented in association with Block S130 can be used to correct for non-linear analyte sensor responses, and to correct for differences between amounts of an analyte within the blood stream in comparison to amounts of the analyte within interstitial fluid. In more detail, the molecular/physiological models used describe one or more of: enzyme kinetics associated with the analyte sensor, diffusive properties of layers or regions (e.g., polymer layers/regions) of the analyte sensor, rates of electrochemical reactions associated with regions of the analyte sensor (e.g., electrochemical reaction rates at a surface of the analyte sensor), and any other suitable factor associated with molecular/physiological behavior of the analyte(s) of interest.

In some variations, the molecular/physiological models can characterize dynamic/kinetic behavior of analytes based upon one or more of: molecular parameters of the analyte molecule(s), geometrical parameters of the analyte molecule(s), electrotopological parameters of the analyte molecule(s), connectivity parameters of the analyte molecule(s), and any other suitable parameter(s) of analytes. In specific examples, the molecular parameters can comprise counts of molecular groups/atom types associated with the analyte(s) of interest, the geometrical parameters can comprise distance-derived characteristics associated with atoms of the analyte(s) of interest, the electrotopological parameters can comprise electrotopological state indices, and the connectivity parameters can comprise chi indices associated with the analyte(s) of interest. However, the parameters of the molecular/physiological models can additionally or alternatively comprise any other suitable parameters.

As shown in the flowchart of FIGS. 3A and 3B, the molecular/physiological models can be used to iteratively refine the calibration operation used in Block S130, and outputs derived from the molecular/physiological models can be stored within the personal database of the user. However, use of the molecular/physiological models in Block S130 can additionally or alternatively be implemented in association with other blocks of the method 100 in any other suitable manner.

As shown in the flowchart of FIG. 3B, diffusion models implemented in association with Block S130 can be used to characterize diffusion behavior of the analyte(s) of interest (e.g., across regions of the analyte sensor, with interstitial fluid, within body fluid, between the blood stream and interstitial fluid, etc.). As shown in the flowchart of FIG. 3B, the diffusion models can be used to iteratively refine the calibration operation used in Block S130, and outputs derived from the diffusion models can be stored within the personal database of the user. However, use of the diffusion models in Block S130 can additionally or alternatively be implemented in association with other blocks of the method 100 in any other suitable manner.

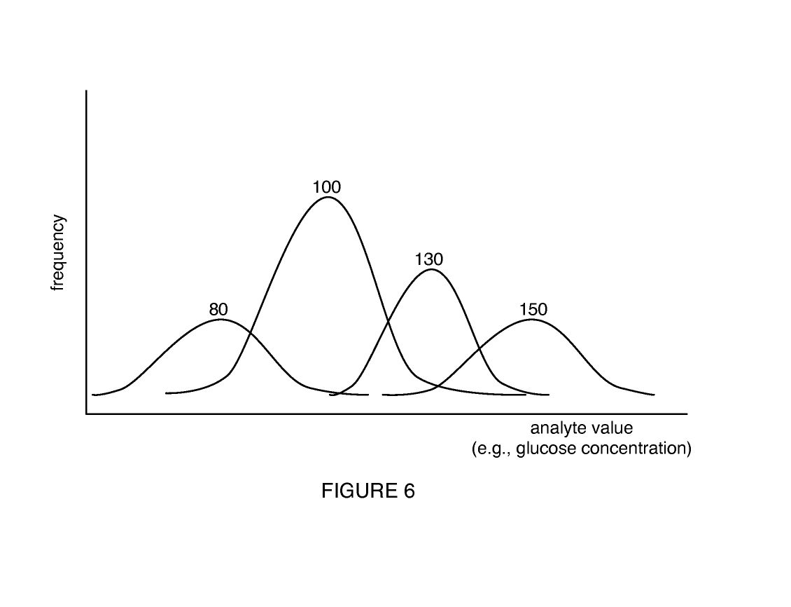

In Block S130, the calibration operation can additionally or alternatively include a Gaussian mixture model, a finite mixture model, and/or a log normal distribution that models the multimodal nature of glucose concentrations (or other analyte concentrations) that can occur over long periods of time. In more detail, a multimodal finite mixture model can operate under the assumption that the body tends to experience analyte levels (e.g., concentrations) at different frequencies, as shown in FIG. 6, according to one or more factors including time of day, consumption of a meal, performance of an exercise activity, and other factors associated with behaviors and/or health states of the user. The distribution of frequencies of different analyte levels can thus be transformed into a state-space model (i.e., a mathematical model of a physical system as a set of input, output, and state variables related by one or more differential equations) for each user, thereby enabling identification of the most probable states of an analyte parameter for a user, given a set of known factors associated with the user's behavior and environment. As such, the state space model can be used to calibrate analyte parameter values determined from the signal stream, without requiring a blood-based measurement from the user for calibration.

Block S130 can, however, additionally or alternatively include any other suitable data-processing method that facilitates calibration. For instance, Block S140 can include implementing a machine-learning algorithm or other algorithm configured to facilitate automatic identification of features (e.g., characteristic physiological responses, outliers) within raw or processed biosignal data, and matching of features in the sensor data with patterns of behavior of one or more users. In variations, regression techniques can also be implemented and can include any one or more of: local regression techniques (e.g., locally segmented regression, B-spline regression, Bezier curve approaches, etc.), regularized regression (e.g., Tikonov regularization, local angle regression, ridge regression, elastic net regression, etc.), nonlinear regressions (e.g., Gaussian regression, Lorentzian regression, Poisson Mixture Models, Artificial Neural Networks, Decision-tree, Random Forest, CART methods, etc.), and any other suitable regression technique. In variations, Block S130 can additionally or alternatively include implementation of time-delay estimations for improved calibration (e.g., using Clarke-error grid analysis, Bland Altman Analysis, using Consensus-based metrics of sensor performance, etc.). Block S130 can additionally or alternatively include auto-calibration techniques, including any one or more of: exploitation of circadian rhythms and other cyclic patterns of the user, exertion and ingestion (e.g., glucose-ingestion) models, machine-learning pattern-classification approaches (e.g., kNN, k-means, support vector machines, gradient descent, self-organized maps, graphical models, etc.), methods relying on quadratic loss functions, and any other suitable method.

Any of the above variations can involve application of any of the above filtering methods. Furthermore, the machine learning algorithm(s) can include any suitable class of machine learning algorithm (e.g., unsupervised learning, semi-supervised learning, transduction, reinforcement learning, etc.) and can additionally or alternatively involve implementation of a data-mining algorithm. Any models produced using a machine learning algorithm can further be trained with data from the user and/or data from any other user(s) to increase the robustness of analyses derived from outputs of Block S130. Furthermore, in Blocks S120 and/or S130, variations of signal processing and calibration models can include implementation of any one or more of: stochastic process models, chaotic models (e.g., models that implement Hurst Exponentials), agent-based and genetic algorithm models, and any other suitable model.

Block S140 recites: generating a set of calibrated values of the analyte parameter as outputs of Block S130. The metric(s) can be indicative of any one or more of: an analyte level (e.g., a concentration of an analyte in body fluid, an absolute amount of an analyte in body fluid), a change in an analyte level (e.g., a rate of change in an analyte level, a difference between analyte levels determined at different time points, a difference between an analyte level and a calibration state analyte level as in a relative analyte measure, etc.), a comparison between metrics for different analytes (e.g., a ratio between a first analyte metric and a second analyte metric), a parameter derived from any of the above parameters, and any other suitable characteristic of one or more analyte levels. In some variations, the metric can be a representative metric characterizing multiple analytes and/or multiple analyte parameters) such that values of a comprehensive body chemistry metric may be generated in Block S140. The set of values can be qualitative and/or quantitative, in characterizing the analyte(s) sensed in Block Silo. For instance qualitative values can be based upon qualitative characterizations of analyte metrics, and quantitative values can provide numerical characterizations of analyte metrics. In one example, the analyte parameters can be associated with one or more of: analytes involving an amperometric measurement (e.g., potassium, sodium calcium, alcohol, cortisol, hormones, etc.), analytes involving a conductometric measurement (e.g., for analytes including glucose, lactic acid, creatinine, etc.), and any other suitable type of analyte detectable in body fluid of the user.

In one variation, the calibration state identified in Block S130 can be used to categorize values of the metric qualitatively within a set of categories, such that the set of calibrated values of the analyte parameter(s) in Block S140 are qualitative values. In one example, a range including analyte levels surrounding values corresponding to the calibration state can be characterized as “normal”, wherein values higher than an upper boundary of the range characterize analyte levels as being “high”, and values lower than a lower boundary of the range characterize analyte levels as being “low”. In variations of the example, qualitative characterization of values of a metric can be determined with any suitable resolution (e.g., low/normal/high, low/borderline low/normal/borderline high/high, etc.). As such, categorization of values of the metric can be performed with any suitable number of categories, wherein the ranges corresponding to different categories can have any suitable range of values. Furthermore, the ranges can be adjustable (e.g., based upon a length of time during which the user interacts with the method 100). In some variations, qualitative categorizations can further be used to derive quantitative values. For instance, quantitative characterizations of durations in which the user spends in a given category can be determined based upon categorical characterizations across the set of time points. The quantitative/qualitative values can be rendered or otherwise provided to the user and/or any other suitable entity, some examples of which are described in U.S. application Ser. No. 14/657,973, entitled “System for Monitoring Body Chemistry” and filed on 13 Mar. 2015, which is herein incorporated in its entirety by this reference. Provision of information derived from the set of calibrated values of the analyte parameter is described in more detail in Section 1.4 below.

### 1.3 Method—Supplemental Data

Block S150 recites: identifying a set of activity events of the user, from a supplemental dataset, during a time window corresponding to the set of time points. Block S150 functions to facilitate identification of associations between a user's activity or behavior and sensed analyte parameters/determined metric values. The supplemental dataset can additionally or alternatively be used, in relation to Blocks S120-S140, to select appropriate parameters of the physiological models, the molecular models, and any other suitable models used in signal processing and calibration, in order to identify and correct artifacts (e.g., spurious values and peaks), based upon information about the user's activity, geographical location, time of day. The supplemental dataset can be derived from sensors integrated in other wearable or mobile computing devices of the user, from application programming interfaces (APIs) configured to provide relevant user data to the computing system associated with the method 100, from user input (e.g., information manually input by the user in a dative application executing at the user's mobile computing device), from electronic health records of the user, from aggregated health information repositories (e.g., Apple Health Kit), from electronic social networks of the user (e.g., Facebook, Twitter, Instagram, etc.), from databases of clinical studies, and/or from any other suitable source. Furthermore, the supplemental dataset can be stored and accessible through the personal database/repository for the user, as described above.

In facilitating identification of activity events of the user in Block S150, the supplemental dataset can include data characterizing physiologically relevant parameters, including any one or more of: electrophysiological signals (e.g., electroencephalogram signals, electromyography signals, galvanic skin response signals, electrocardiogram signals, etc.), body temperature, respiration parameters (e.g., respiration rate, depth/shallowness of breath, etc.), blood flow parameters, pulse oximetry signals, motion parameters, and any other suitable physiologically relevant parameter of the user. Additionally or alternatively, physiologically relevant cycles, as detected using a biorhythm or biogram cycle database (e.g., derived from user inputs, or determined by an algorithm that tracks the user's physical, emotional, or neurological states corresponding to a detected single or multiple analyte) can contribute to the supplemental dataset. The supplemental dataset can additionally or alternatively include data related to an environment of the user, such as a location of the user (e.g., as determined from a global positioning device, from a triangulation device), environmental temperature of the user, environmental audio of the user, and any other suitable environmental information of the user.

As such, identifying the activity event from the supplemental dataset in Block S150 can include identifying an activity associated with one or more of: eating (e.g., based upon sensor data, based upon updates within electronic social networks, based on GPS location, etc.), drinking (e.g., based upon sensor data, based upon updates within electronic social networks, etc.), substance use (e.g., based upon sensor data, based upon updates within electronic social networks, based on GPS location, based on electronic health record information, etc.), exercise (e.g., based upon biosignal sensor data, based upon updates within electronic social networks, based on GPS location, etc.), health or disease-related events (e.g., based upon biosignal sensor data, based upon information extracted from an electronic health record, etc.), psychological state (e.g., based upon biosignal data, based upon updates within electronic social networks, etc.), and any other suitable event.

In some variations, the supplemental dataset can include demographic information of the user, including information related to any one or more of: gender (e.g., male, female, etc.), age, weight, height, ethnicity, marital status, profession, geographic location, diagnosed medical conditions (e.g., diabetes, alcohol intolerance), metabolic profile (e.g., fat/muscle content), family history, genetic information, and any other suitable type of demographic-related information. As such, the event(s) characterized in the supplemental dataset can include any one or more of: physiological events, behavior events (e.g., events related to eating, drinking, sleeping, resting, exercising, etc.), location events (e.g., changes in a user's location), and any other suitable event captured in the supplemental dataset.

The supplemental dataset can be received manually (e.g., based upon user input of events), or can additionally or alternatively be received automatically. In one variation of manual reception, receiving the supplemental dataset in Block S150 can include allowing a user to input an event, which can be implemented by a software module with a user interface, executing on an electronic device that allows the user or another entity is able to input an activity using the software module. In the example, a user may input at least one of an event related to a meal, an event related to exercise or rest, and an event related to a medication or nutritional supplement. In variations of the example of manual reception, Block S150 can further comprise allowing a user to input a temporally varying user characteristic (e.g., emotional or physical state), or substantially invariable user characteristic (e.g., gender or demographic). In one variation of automatic reception, at least a portion of the supplementary dataset can be received using a supplementary sensing module configured to generate the supplemental dataset. In another variation of automatic reception, at least a portion of the supplementary dataset can be received using a module configured to extract information from networks (e.g., social networks, personal networks) of the user.

The activity event(s) captured in the supplemental dataset is/are preferably directly associated with at least one time point of the set of time points over which the signal stream is captured in Block S110, in order to facilitate generation of the association in variations of Block S160, as described below. As such, upon implementation of Block S150, data of the biosignal dataset can be tagged with user activity event information, based upon events captured in the supplemental dataset and the set of time points. In some variations, however, sensing of the analyte parameter(s) in Block S110 can include an inherent sensing delay based, for instance, upon a delay between ingesting a consumable and detection of the consumable in the body fluid (e.g., interstitial fluid, blood) of the user. As such, some variations, of Block S150 can include associating an event of the user, captured in the supplemental dataset, with a past or future time point of the set of time points, based upon a delay between the event and detection as in Block S110. The event(s) of the supplemental dataset can, however, correspond to the time points of the biosignal dataset in any other suitable manner.

### 1.4 Method—Analyses, Interpretation, and Insight Provision

Block S160 recites: generating an analysis indicative of an association between at least one of the set of activity events and the set of calibrated values of the analyte parameter. Block S160 functions to facilitate formation of insights between events of the user and physiological responses represented in body analyte parameters, in a manner that is meaningful to a user, and as relevant to different time scales (e.g., minutes, weeks, months, years, etc.). As such, Block S160 can be used to characterize and identify trends associated with behaviors of the user and their physiological responses as captured in the analyte data. In Block S160, calibrated analyte time series data for the user can thus be analyzed in the context of historical behavior of the user, personal information of the user, contextual information of the user (e.g., information pertaining to the user's environment, activity of the user, etc.), and other information of the user. Furthermore, the analysis generated in Block S160 can be compared to analyses associated with a population of users, such that the user is provided with insights derived from population-wide data. As described in more detail below, the analysis generated in Block S160 can be used for one or more of: providing actionable feedback to the user for maintaining healthy analyte (e.g., glucose) levels, predicting trends in user behavior, modifying trends in user behavior, providing information derived from the combination of analyte parameter values and user activity (e.g., a measure of caloric intake).

Preferably, in Block S160, the identified activity event(s) is/are correlated with the value(s) of the analyte parameter(s) determined at time points associated with the event(s) captured in Block S150, such that direct correlations can be made between events of the user and values of the analyte parameter(s) over time. However, any other suitable association between an activity event of the supplemental dataset and values of metrics from the biosignal dataset can be generated in Block S160. For instance, an event (e.g., ingestion of a slow-digesting consumable, heavy physical activity) that is significantly separated in time from that of a physiological response can be associated with values of a metric at a distant time point of the set of time points. In another example, biosignal data associated with a range of time points can be associated with an event captured in the supplemental dataset. In one implementation of this example, a value of a parameter (e.g. glucose level, analyte level, comprehensive metric value) is associated with an event (e.g., meal), using data from time points spanning the meal and a period of time after the meal.

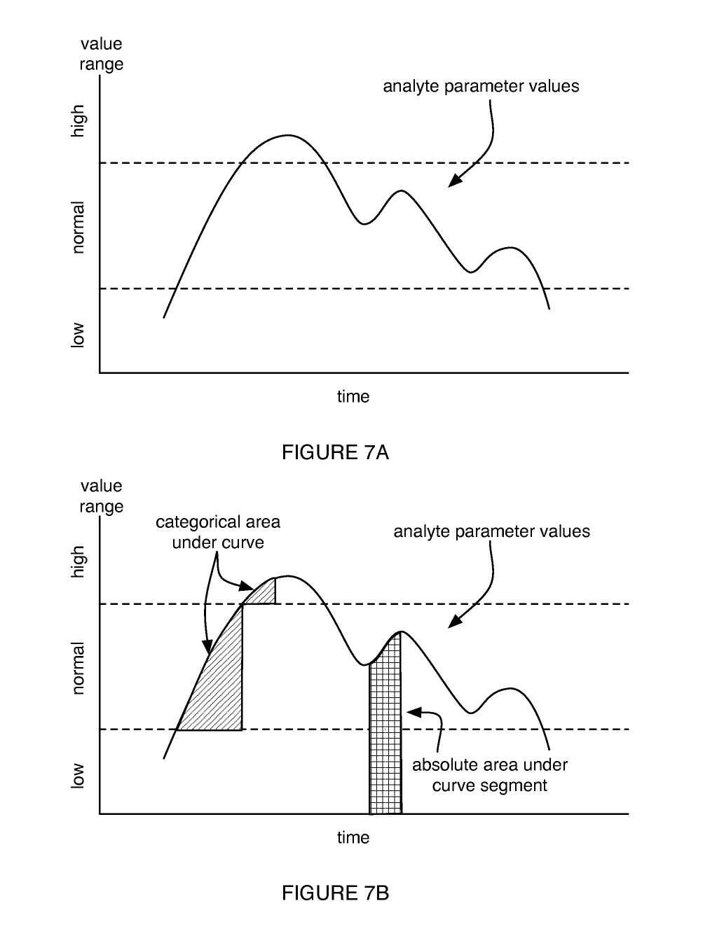

In Block S160, the analysis can additionally or alternatively provide user-specific responses to different activity events (e.g., a food-related event, an exercise-related event, etc.). In one such variation, a shape of a curve generated from the signal stream/set of calibrated values of an analyte parameter and the set of time points can be used generate derivative measurements indicative of a user physiological response to an activity event, as shown in FIG. 7A. In particular, a rate of change of an analyte parameter captured in a slope of the curve, and an area under the curve (e.g., an area under the entire curve, an area under a portion of the curve) can be used to generate derivative measurements in the analysis of Block S160. In one variation, the curve can be divided into a set of segments (e.g., based upon ranges of values, based upon the set of time points), wherein the area under the curve for a given segment can be used to generate derivative measurements as shown in FIG. 7A. In another variation, as shown in FIG. 7B, an area under a curve (e.g., for a segment of the curve, for the entire curve) within a specific range of time points can be used to generate derivative measurements of the analysis of Block S160.

In one specific example, an area under a curve of calibrated glucose concentration values can be characteristic of consumables ingested by the user. For instance, an area under the curve of calibrated glucose concentration curve (e.g., up to a time point, between time points) can be indicative of a glycemic index of food ingested by the user. As such, one or more areas under the curve can be used to derive a “personalized” glycemic index for consumables ingested by the user. Thus, the analysis of Block S160 can generate a personalized glycemic load (or other analyte-related “load” parameter) associated with a particular food that the user consumes. However, any other suitable shape parameter(s) of the curve (e.g., peak durations, plateau durations, peak amplitudes, number of peak instances, number of peak irregularities, number of inflection points, location of curve elements, etc.) can be used to generate derivative measurements in the analysis of Block S160, such as a characteristic time over which the user returns to a baseline analyte parameter state post experiencing an activity event (e.g., consumption of a particular type of food, performance of a specific type of exercise, etc.). Furthermore, the derivative measurements can include relative measurements and/or absolute measurements.

In Block S160, the analysis can additionally or alternatively provide user-specific physiological states as determined in an unconventional manner. For instance, in one variation, information determined from multiple sensor streams (e.g., analyte sensor streams, biosignal sensor streams, supplemental datasets) and physiological models can be used to extract indirectly measured biophysical properties. In a specific example, Block S160 can comprise determining caloric flow (e.g., caloric intake, caloric expenditure) based upon a model that processes one or more calibrated glucose concentration values, body temperature, and exercise activity to determine a current state of caloric intake/expenditure of the user. Determination of user-specific physiological states can, however, be determined based upon any other alternative or additional parameter(s) associated with different analytes of interest.

In some variations, information from the analysis can be transmitted to an API endpoint, as shown in FIGS. 3A and 3B, which can be accessed by one or more of: an application executing on a mobile computing device (e.g., a mobile communication device, a wrist-borne mobile computing device, a head-borne mobile computing device, etc.), a web browser-based application, and any other suitable software application (e.g., a software application of a client associated with one of the user and the computing system implementing the method 100). As such, the API endpoint can interface with applications external to the computing system implementing portions of the method 100, in providing access to analyte-derived data, analyses that correlate analyte data with other sensor streams, and conclusions/predictions derived from the analyses of Block S160.

Block S170 recites: rendering information derived from the analysis to the user, which functions to generally inform the user and/or provide a mechanism to promote behavior change or maintenance of beneficial behaviors in relation to identified associations between the user's behavior and associated physiological states. Block S170 can comprise generating and providing a user notification based on the analysis of Block S160, wherein the user notification preferably contains information relevant to a physiological state of the user, as captured in values of analyte parameters determined from the biosignal dataset, at least at one time point of the set of time points. The notification can be configured to suggest an action that the user can perform to rectify an undesired physiological state indicated by the value(s) of the metric(s) corresponding to one or more time points of the set of time points and related to the analysis generated in Block S160. For instance, the notification can provide a suggestion to the user to go for a walk post-consumption of a meal, or upon detection of a spike in values of an analyte parameter (e.g., a glucose concentration parameter). In another example, the notification can provide a suggestion to the user to consume a snack, upon detection of a low value of an analyte parameter (e.g., a low glucose concentration state). Additionally or alternatively, the notification can be configured to promote maintenance of a desired physiological state indicated by the value(s) of the metric(s) corresponding to one or more time points of the set of time points. In one example, the notification can provide a suggestion to the user to continue exercising in order to decrease a high value of an analyte parameter (e.g., a glucose concentration parameter).

The notification can additionally or alternatively be configured to inform the user of a trend in values of an analyte parameter (or derivative thereof) indicative of a user's current or anticipated future physiological state, as described further in relation to Block S180 below. For instance, the notification can alert the user of one or more of: a high value of an analyte parameter, a low value of an analyte parameter, a dramatic trend toward high values of an analyte parameter at a future time point, a dramatic trend toward low values of an analyte parameter at a future time point, and any other suitable anticipated undesired state indicated in the value(s) of the metric(s). In another example, the notification can inform the user regarding durations of time in which the user is in a “low” state of an analyte parameter, a “normal” state of the analyte parameter, and a “high” state of the analyte parameter, with the goal of motivating the user to achieve 100% normalcy in values of the metric. In furthering this example, the range of values corresponding to the “normal” state can be adjustable to adjust the difficulty in achieving 100% normalcy in values of the metric.

The notification can additionally or alternatively be configured to provide insights into patterns in the user's behaviors and physiological responses, based upon the association(s) generated in Block S160. In one example, the notification can provide insights into daily trends in the user's behavior, and positive/negative effects of eating or exercising at given times in a routine of the user. In another example, an association between eating at a given dining facility and a subsequent analyte level (e.g., glucose level), as determined in a variation of Block S160, can be used to provide a notification to the user that encourages or discourages eating at the dining facility. In another example, an association between eating a specific food item and an associated value of a metric (e.g., analyte level metric, energy level metric, mental state characterization metric, etc.), as determined in a variation of Block S160, can be used to provide a notification to the user that encourages or discourages eating the specific food item. In another example, an association between eating at an unusual time (in relation to a routine of the user) and an associated value of a metric (e.g., analyte level metric, energy level metric, mental state characterization metric, etc.), as determined in a variation of Block S160, can be used to provide a notification to the user that discourages eating off-schedule. As such, the notification can provide insights into associations between the user's consumption, activities, and or mental states in a meaningful manner. Furthermore, the user can gain insights into his/her phenotype, with regard to metabolic activity, based upon the notification(s) provided in Block S170.

The notification of Block S170 can be any one or more of: a visual notification, an audio notification, a haptic notification, and any other suitable notification. The notification is preferably provided at an electronic device (e.g., mobile device, personal computer, wearable computing device, tablet, etc.) of the user, but can additionally or alternatively be provided to the user in a non-electronic manner. In one variation, the notification can be provided at a messaging client (e.g., text messaging client, email client, etc.), accessible by the user at a mobile device and/or a personal computer of the user. In another variation, the notification can be provided using a vibration motor of an electronic device of the user. However, the notification can be provided in any other suitable manner, embodiments, variations, and examples of which are described in U.S. application Ser. No. 14/657,973, entitled “System for Monitoring Body Chemistry” and filed on 13 Mar. 2015. Furthermore, in some variations, the notification can additionally or alternatively be provided to an entity associated with the user, in order to inform another entity of a state of the user. In variations, the entity can be any one or more of: a parent, a sibling, a significant other, a healthcare provider, a supervisor, a peer, and any other suitable entity associated with the user. As such, the entity can facilitate motivation of the user to adjust a behavior to improve his/her physiological state.

In relation to a set of possible notifications that can be provided to the user, classification of the set of notifications according to priority can be used to govern notification provision. Thus, in some variations, notifications can be prioritized or otherwise ranked according to severity (or any other suitable factor), which can be used to prioritize and/or limit the number of notifications provided to a user. In variations, the number and/or type of notifications provided to the user can be governed based upon user preference or any other suitable factor. In one example of notification ranking, notifications associated with events that have a near-term (e.g., immediate) adverse impact on the user's wellbeing can be elevated to a higher priority level (e.g., “imminent sugar crash—eat something healthy soon”). In the example, notifications associated with events that are moderately severe but not urgent can be given a medium priority level (e.g., “that meal was larger than your average lunch—consider going for a walk”). Finally, in the example, informational notifications are given the lowest priority level (e.g., “You spent ˜80% of today in the green zone. Well done!”). In the example, a user can elect to receive all notifications, or can alternatively elect to receive only a subset of notifications generated (e.g., upon submission of user preferences at an input device in communication with the system). Furthermore, in relation to notification priority, a user can modify priority rankings of one or more types of notifications. For instance, a user can elevate the priority level of a notification associated with an event, in order to always be notified whenever the event is detected. In more detail, a user can elect to always receive an informational notification (e.g., a notification summarizing daily performance of the user), which would otherwise have a low priority ranking, thereby elevating informational notifications to a higher priority level.

As shown in FIG. 1, the method 100 can further include Block S180, which recites: determining an anticipated future state of the user based upon values of the analyte parameter, the set of activity events, and the set of time points. Block S180 functions to form additional insights based upon historical biosignal data of the user that can be used to predict future physiological states of the user in relation to the user's behavior. Block S180 preferably implements historical data of the user in the prediction; however, Block S180 can additionally or alternatively implement data of any other user(s) in the prediction. Block S180 can include implementing a machine learning algorithm configured to facilitate automatic identification of correlations between repeated activity events and physiological responses of the user within raw or processed biosignal data (e.g., in signal signatures, in values of analyte parameters, etc.), which can be used to facilitate identification of causal relationships between events and physiological states of the user. The machine learning algorithm(s) can include any suitable class of machine learning algorithm (e.g., unsupervised learning, semi-supervised learning, transduction, reinforcement learning, etc.) and can additionally or alternatively involve implementation of a data-mining algorithm. The algorithms used to determine the anticipated future state of the user can additionally or alternatively implement a probabilistic analysis that generates a set of candidate future states and ranks the candidate future states according to probability of occurrence. However, the anticipated future state of the user can additionally or alternatively be determined in any other suitable manner.

In some variations, determining the anticipated future state of the user can be based upon an identified repetition of at least one activity event captured by the supplemental dataset, which have been historically associated with values of the analyte parameters and/or signal signatures identified within the signal stream. The anticipated future state can be based upon occurrence (or lack thereof) of a single activity event, or can additionally or alternatively be based upon a combination of a set of identified activity events that occur in a specific pattern (e.g., in sequence, simultaneously). As such, in one variation, an anticipated future state can be based solely upon identification of a specific activity event (e.g., an activity event associated with eating, exercise, etc.), and in another variation, an anticipated future state can be based upon a pattern of activity events (e.g., the user has not eaten, has consumed little liquid, and has undergone a period of heavy exercise).

In variations, the values of the analyte parameter(s) and their trajectories (e.g., actual trajectories, anticipated trajectories, etc.), in combination with additional sensor signal streams from the supplemental dataset can be used to make near and long-term predictions. In more detail, the predictions can be based upon identification of unique signal signatures within the calibrated signal stream of Block S130, and correlation of these unique signal signatures both physiological models of the user and activity of the user. As such, in one specific example of a near-term prediction, the anticipated future state can be used to determine if/when the user will be in a “low” state or a high state of a analyte parameter, based upon any one or more of: a time point at which the user ate, historical signal signatures characterizing values of the analyte parameter(s) over time in relation to repeated eating events, a specific type of meal, a specific time of a meal, an amount of exercise performed and repeated by the user, the type of exercise performed and repeated by the user, a repeated physical location of the user, and any other suitable repeated event of the user. As such, the specific example can indicate if the user is anticipated to enter a hypoglycemic state or a hyperglycemic state, based upon one or more of a set of behaviors of the user. In a related example, the anticipated state can indicate that the user has not eaten and will enter an agitated state associated with hunger (e.g., will become “hangry”) based upon a lack of food consumption and increased stress (e.g., from other biosignal data).

In one specific example of a long-term prediction, the anticipated future state can be used to determine if/when the user will trend toward a given disease state (e.g., a state of diabetes, a state of obesity, a state associated with a thyroid disorder, a state associated with a cardiovascular disorder, etc.), without correction of behavior, based upon one or more of: repeated signal signatures associated with analyte parameter states/trajectories, detection of repeated poor diet events, detection of repeated lack of exercise events, detection of repeated stress-related events, and any other suitable activity event or signal signature. The anticipated future state can additionally or alternatively be based upon analysis and detection of general changes in analyte behavior (e.g., blood glucose behavior) over a longer duration of time (e.g., a change in the time required for a blood glucose concentration to return to a baseline value after an event, a change in the baseline blood glucose concentration of a user over a long duration of time, etc.).

In variations, Block S180 can additionally or alternatively account for one or more boundary conditions provided by the user or another entity. For instance, a prediction of an anticipated state of the user, indicated in values of analyte parameter(s), can be determined based upon knowledge that the user intends to perform an event in the future, that the user has specific goals for his/her health, and/or based upon any other suitable knowledge. For instance, generating a prediction of an anticipated state can be based upon knowledge (e.g., provided by user input) that the user will begin physical exercise at a future time point, which can be used to predict a falling value of an analyte parameter characterizing the user's physiological state. In another example, generating of a prediction of an anticipated state can be based upon knowledge that the user has a goal of burning calories, which can be used to predict future ranges of values of an analyte parameter based upon anticipated behaviors of the user. In another example, generating a prediction of an anticipated state of the user can be based upon detection of an approaching event of the user, which can be used to predict a value of an analyte parameter characterizing the user's physiological state. In this example, detection of the approaching event can be based upon detection of the user's location (e.g., the user is approaching a diner, the user is approaching an exercise facility) or detection of any other activity of the user. The boundary conditions can, however, include any other suitable conditions provided by the user or another entity, which would affect modeling anticipated physiological states of the user.

Furthermore, in relation to Block S170, a notification can be provided to the user based upon the anticipated state determined in Block S180. The notification can be configured to recommend an activity to the user, wherein performance of the activity by the user will prevent the anticipated state from occurring (i.e., if the anticipated state is an adverse state), or cause the anticipated state to occur (i.e., of the anticipated state is a positive state).

In one example, a notification can be provided to the user based upon a prediction that the user will enter a “low” state, wherein the notification prompts the user to eat a snack prior to (e.g., 20 minutes before) an anticipated low state (e.g., a “hangry” state). In this example, the notification thus provides a recommended activity that should cause the user to return to a healthy state. In another example, a notification can be provided to the user based upon a prediction that the user will enter a “high” state, wherein the notification prompts the user to exercise prior to an anticipated high state. In another example, a notification can be provided to the user based upon a condition that the user will begin physical activity at a future time, wherein the notification prompts the user to eat at a specified time prior to the time of physical activity. In another example, a notification can be provided to the user based upon knowledge of a calorie-burning goal of the user, wherein the notification informs the user regarding times of day during which the user's exercising will be most effective. In another example, a notification can be provided to the user based upon detection of an approaching event of the user (e.g., the user has approached a diner), wherein the notification informs the user regarding his/her past physiological responses subsequent to past occurrences of the event (e.g., physiological responses to eating at the diner). The notification can, however, include any other suitable notification. Additionally, variations and examples of provision of information derived from anticipated analyte level trajectories, in association with the anticipated future state of the user, are described in U.S. application Ser. No. 14/657,973, entitled “System for Monitoring Body Chemistry” and filed on 13 Mar. 2015. In particular, information derived from the anticipated future state/analyte level trajectories can be provided within an application executing at a computing device (e.g., mobile computing device, wearable computing device, web browser-based application, etc.) of the user.

Again, and similar to the methods described above in Block S170, classification of anticipated future states according to priority can be used to govern notification provision. Thus, in some variations, anticipated future states determined according to Block S180 can be prioritized according to severity (or any other suitable factor), which can be used to prioritize and/or limit the number of notifications provided to a user. In variations, the number and/or type of notifications provided to the user can be governed based upon user preference or any other suitable factor.

The method 100 can, however, include any other suitable blocks or steps that facilitate detection, processing, and/or analyzing of biosignals of the user in a manner that provides insights into the user's physiological states.

The FIGURES illustrate the architecture, functionality and operation of possible implementations of systems, methods and computer program products according to preferred embodiments, example configurations, and variations thereof. In this regard, each block in the flowchart or block diagrams may represent a module, segment, step, or portion of code, which comprises one or more executable instructions for implementing the specified logical function(s). It should also be noted that, in some alternative implementations, the functions noted in the block can occur out of the order noted in the FIGURES. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The method 100 of the preferred embodiment can be embodied and/or implemented at least in part as machine configured to receive a computer-readable medium storing computer-readable instructions. The instructions are preferably executed by computer-executable components preferably integrated with the system and one or more portions of the processor and/or analysis engine. The computer-readable medium can be implemented in the cloud, and/or stored on any suitable computer-readable media such as RAMs, ROMs, flash memory, EEPROMs, optical devices (CD or DVD), hard drives, floppy drives, or any suitable device. The computer-executable component is preferably a general or application specific processor, but any suitable dedicated hardware or hardware/firmware combination device can alternatively or additionally execute the instructions.

As a person skilled in the art will recognize from the previous detailed description and from the figures and claims, modifications and changes can be made to the preferred embodiments of the invention without departing from the scope of this invention defined in the following claims.

We claim:

1. A method for near-real time and continuous analyte monitoring, the method including:
at an analyte sensor in communication with interstitial fluid of a user, receiving a signal stream associated with an analyte parameter of the user across a set of time points;
at a computing system in communication with the analyte sensor, generating a dataset indicative of values of the analyte parameter across the set of time points, upon processing of the signal stream in near-real time;
performing a calibration operation on values of the analyte parameter, based upon a calibration event unassociated with a blood-sampled measurement of the user, thereby generating a set of calibrated values of the analyte parameter;
at the computing system, identifying a set of activity events of the user, from a supplemental dataset, during a time window corresponding to the set of time points;
generating an analysis indicative of an association between at least one of the set of activity events and the set of calibrated values of the analyte parameter; and
at a mobile computing device associated with the user and in communication with the computing system, rendering information derived from the analysis to the user.

2. The method of claim 1, wherein receiving the signal stream associated with the analyte parameter includes receiving the signal stream associated with a glucose parameter.

3. The method of claim 1, wherein performing the calibration operation comprises detecting a sleep-associated state of the user as the calibration event, and using a fasted state related to the sleep-associated state of the user to generate the set of calibrated values of the analyte parameter.

4. The method of claim 1, wherein performing the calibration operation comprises prompting the user to ingest of substance configured to produce a known physiological response in the user as the calibration event, and generating the set of calibrated values of the analyte parameter based upon the known physiological response.

5. The method of claim 1, wherein performing the calibration operation includes implementing a molecular model that characterizes at least one of: enzyme kinetics associated with the analyte sensor, diffusive properties of regions of the analyte sensor, and rates of electrochemical reactions associated with regions of the analyte sensor.

6. The method of claim 1, wherein processing the signal stream includes detecting an artifact within the signal stream in near-real time, and characterizing the artifact as one of a short duration artifact and a long duration artifact.

7. The method of claim 6, wherein characterizing the artifact comprises at least one of: 1) associating the short duration artifact with physical disturbance of the glucose sensor, and 2) associating the long duration artifact with at least one of a) sensor equilibration upon application of the analyte sensor to the user and b) sensor drift.

8. The method of claim 1, wherein identifying the set of activity events includes identifying a consumption event of the user, and wherein generating the analysis includes: generating a curve of values of a glucose parameter over time, determining an area under a portion of the curve associated with the consumption event, and determining a personalized glycemic load of the consumption event based upon the area under the portion of the curve.

9. The method of claim 1, wherein generating the analysis includes determining a caloric flow state of the user from the set of calibrated values of the analyte parameter, body temperature of the user from the supplemental dataset, and exercise activity of the user from the supplemental dataset.

10. The method of claim 1, wherein rendering information derived from the analysis to the user includes notifying the user, at the mobile computing device, of an activity that the user can perform to achieve an improved health state.

11. A method for continuous analyte monitoring, the method including:
at a sensor in communication with body fluid of a user, receiving a signal stream associated with an analyte parameter of the user across a set of time points;
at a computing system in communication with the sensor, generating a dataset indicative of values of the analyte parameter across the set of time points, upon processing of the signal stream;
performing a calibration operation on values of the analyte parameter, based upon a calibration event automatically detected within the signal stream, thereby generating a set of calibrated values of the analyte parameter;
at the computing system, identifying a set of activity events of the user, from a supplemental dataset, during a time window corresponding to the set of time points;
at the computing system, determining an anticipated future state of the user, based upon the set of calibrated values of the analyte parameter, the set of activity events, and the set of time points; and
at a mobile computing device associated with the user and in communication with the computing system, recommending an activity to the user, based upon the anticipated future state.

12. The method of claim 11, wherein performing the calibration operation comprises automatically detecting a sleep-associated state of the user as the calibration event, such that the calibration event is unassociated with a blood-sampled measurement of the user, and using a fasted state related to the sleep-associated state of the user to generate the set of calibrated values of the analyte parameter.

13. The method of claim 11, wherein receiving the signal stream includes receiving the signal stream from a glucose sensor configured to interface with interstitial fluid of the user.

14. The method of claim 11, wherein performing the calibration operation includes implementing a molecular model that characterizes at least one of: enzyme kinetics associated with the analyte sensor, diffusive properties of regions of the analyte sensor, and rates of electrochemical reactions associated with regions of the analyte sensor.

15. The method of claim 14, wherein performing the calibration operation includes implementing a diffusion model that characterizes diffusion behavior of an analyte associated with the analyte parameter.

16. The method of claim 11, wherein identifying the set of activity events of the user includes identifying at least one of a consumption event, an exercise event, and a location-based event from the supplemental dataset, and wherein the method further includes generating an analysis indicative of an association between at least one of the set of activity events and the set of calibrated values of the analyte parameter.

17. The method of claim 16, wherein generating the analysis includes determining a caloric flow state of the user from the set of calibrated values of the analyte parameter, and body temperature of the user from the supplemental dataset.

18. The method of claim 11, wherein determining the anticipated future state of the user includes generating a near-term prediction of a low state of the analyte parameter in association with a lack of food consumption by the user, and wherein recommending the activity includes prompting the user to consume a meal to prevent the low state of the analyte parameter.

19. The method of claim 11, wherein determining the anticipated future state of the user includes generating a long-term prediction that the user is trending toward a disease state including at least one of a state of diabetes, a state of obesity, a state associated with a thyroid disorder, and a state associated with a cardiovascular disorder.

20. The method of claim 19, wherein generating the long-term prediction includes tracking change in a time duration required for a blood glucose concentration, of the set of calibrated values of the analyte parameter, to return to a baseline concentration after a consumption event of the set of activity events.
